(19) United States
(12) Patent Application Publication
(10) Pub. No.: US 2011/0245813 A1
(43) Pub. Date: Oct. 6, 2011

(54) TREATMENT TOOL

(76) Inventors: Megumi KIMURA, Tokyo (JP); Keita SUZUKI, Tokyo (JP)

(21) Appl. No.: 13/160,608

(22) Filed: Jun. 15, 2011

Related U.S. Application Data
(63) Continuation of application No. PCT/JP2009/070610, filed on Dec. 9, 2009.

(30) Foreign Application Priority Data
Dec. 16, 2008 (JP) P2008-319789

(51) Int. Cl. A61B 17/94

(52) U.S. Cl. 606/1

(57) ABSTRACT

A treatment tool of the invention includes: a treatment part that can swing about a predetermined swing axis in order to perform treatment to a biological tissue; a plate-like wire-connecting part provided at the treatment part, the wire-connecting part having a predetermined thickness and a through hole formed therein in its thick direction; a linear swing manipulation wire, on one end side whereof are formed a first contacting part that is inserted into the through hole and contacts one face of said wire-connecting part in the thickness direction thereof, and a second contacting part that contacts another face of said wire-connecting part in the thickness direction thereof; a securing member, secured on said one end side of said swing manipulation wire, that holds the first contacting part and the second contacting part such that the first contacting part slides along the face, and the second contacting member slides along the other face; and a manipulating part, provided at another end of the swing manipulation wire, that extends and retracts the swing manipulation wire to make the treatment part swing.

### TECHNICAL FIELD

The present invention relates to a treatment tool, particularly the treatment tool for endoscope. The present application is a U.S. Continued Application based on Patent Application 2008-319789 filed in Japan on Dec. 16, 2008, and on PCT/JP2009/070610 filed internationally in Japan on Dec. 9, 2009, and claims priority on said applications, the contents whereof are quoted herein.

### BACKGROUND ART

A conventional treatment tool for performing treatment such as surgery on a biological tissue includes a treatment part that performs treatment and a manipulation wire for activating the treatment part connected to the treatment part. Particularly in the case of a treatment tool for treating biological tissue through an endoscope, a surgeon performs a remote-controlled operation using a wire that extends from his hand to the treatment part.

As an example of such a treatment tool, Patent Document 1 discloses a treatment tool for endoscope. The treatment tool for endoscope disclosed in Patent Document 1 includes: an associating member inserted via an endoscope into a human body; a treatment part that has a rotatably supported treatment body and is disposed at the distal end of the associating member; a manipulation wire that is extendably and retractably inserted into the associating member; and a link wire connected between the manipulation wire and the treatment body. The link wire is inserted into a connecting hole provided in the treatment body, and includes a connecting part for stopping both sides of a hook part inserted into the connecting hole, thereby forming an annular part.

According to the treatment tool for endoscope described in Patent Document 1, the link wire is used, and the link wire can easily be connected to the connecting hole in an association lever of the treatment body. Moreover, the annular part of the link wire restricts the movement of the hook part with respect to the connecting hole, whereby the opening and closing movements of the manipulation wire performed by a manipulation part can be reliably transmitted to the association lever.

[Patent Document 1] Japanese Patent Application, First Publication No. H11-178829

### SUMMARY OF THE INVENTION

### Problems to be Solved by the Invention

It is an object of the present invention to provide a treatment tool that enables reliable transmission of force applied from the wire to the treatment tool.

### Means for Solving the Problems

To solve the above problems, the invention proposes the following means.

A treatment tool of the invention includes: a treatment part that can swing about a predetermined swing axis in order to perform treatment to a biological tissue; a plate-like wire-connecting part provided at the treatment part, the wire-connecting part having a predetermined thickness and a through hole formed therein in its thick direction; a linear swing manipulation wire, on one end side whereof are formed a first contacting part that is inserted into the through hole and contacts one face of the wire-connecting part in the thickness direction thereof, and a second contacting part that contacts another face of the wire-connecting part in the thickness direction thereof; a securing member, secured on the one end side of the swing manipulation wire, that holds the first contacting part and the second contacting part such that the first contacting part slides along the face, and the second contacting member slides along the other face; and a manipulating part, provided at another end of the swing manipulation wire, that extends and retracts the swing manipulation wire to make the treatment part swing.

According to the invention, since the treatment part and the swing manipulation wire are rotatably linked in the through hole formed in the wire-connecting part, the treatment part swings about the swing axis in association with the extension and retraction of the swing manipulation wire. Also, since the swing manipulation wire is being held by the securing member, the extension and retraction of the swing manipulation wire transmits a dragging/pushing force that makes the securing member also extend and retract. That is, the dragging/pushing force of the swing manipulation wires is evenly transmitted via each swing manipulation wire, and the wire-connecting part is dragged or pushed by an even force. As a result, the swing manipulation wire can be prevented from being displaced from its axis with respect to the through hole in the wire-connecting part, and the treatment part can be swung optimally.

Preferably, in the treatment tool of the invention, the swing manipulation wire is folded back by bending it at both ends of the through hole, and a first insertion hole, which either one of the folded-back sections of the swing manipulation wire is inserted into, and a second insertion hole, which another of the folded-back sections of the swing manipulation wire is inserted into, are formed in the securing member.

In this configuration, since a swing manipulation wire is inserted into each of the first insertion hole and the second insertion hole, the swing manipulation wires can be supported at predetermined distances.

Preferably, in the treatment tool of the invention, the securing member includes a limiting member that is fitted over the swing manipulation wire and supports it.

In this configuration, since the swing manipulation wires can be covered with the limiting members (securing members) and thereby supported collectively, the configuration becomes simple and manufacture becomes easy.

Preferably, in the treatment tool of the invention, the treatment part includes a pair of gripping parts that open and close by swinging about the swing shaft.

In this configuration, since the pair of gripping parts open and close by swinging about the swing shaft, a target object can be gripped by swinging them from the open state to the closed state. Furthermore, since each of the pair of gripping parts swings, their open angle can be increased.

Preferably, the treatment tool of the invention further includes a sheath which the swing manipulation wire is extendably and retractably inserted into, and a distal-end member that is provided between the sheath and the treatment part and connects the distal end of the sheath and the treatment part. Preferably, the distal-end member includes a slit part on at least the distal-end side, and the securing member supports the swing manipulation wire on an inner side of the slit part.

In this configuration, since the securing member is disposed inside the slit part of the distal-end member, the securing member does not catch on the distal-end member or the sheath when the swing manipulation wire is extended and retreated (does not obstruct the retreat of the swing manipulation wire), achieving optimal operation.

### EFFECTS OF THE INVENTION

According to the treatment tool of the invention, by making a treatment part swing by dragging or pushing the parallel parts extending from both ends of a rotation shaft, the force applied from the wires to the treatment d tool can be reliably transmitted.

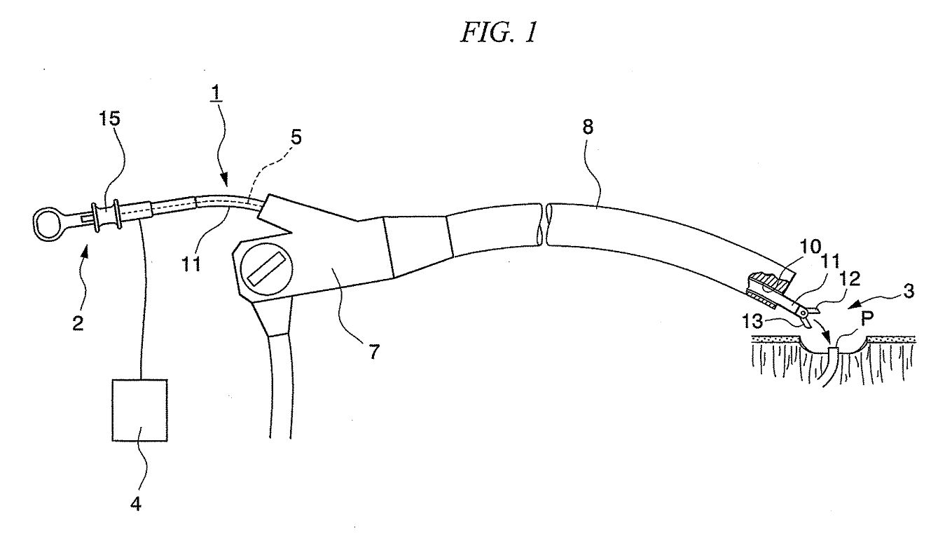

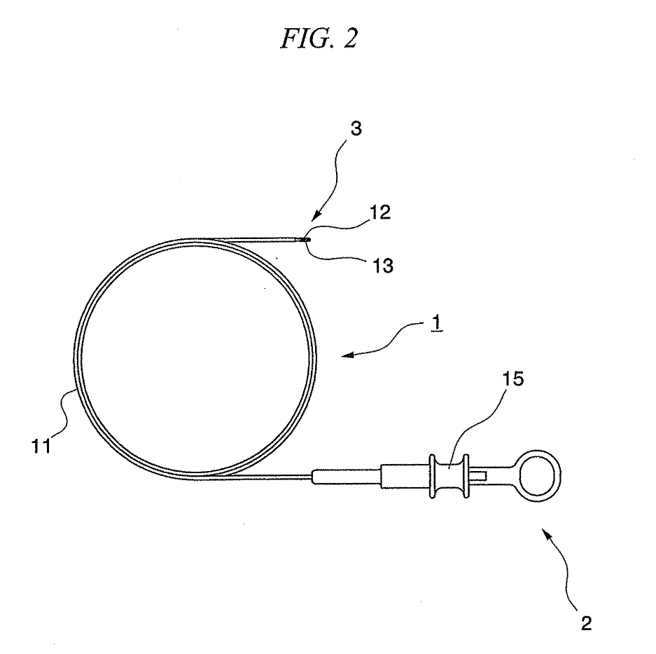

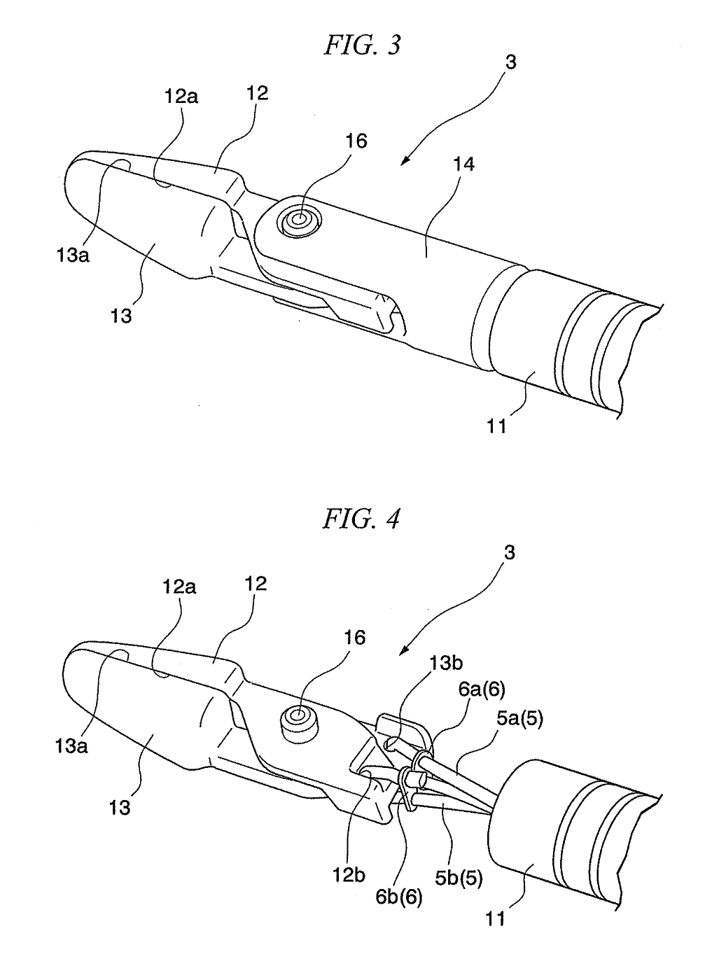

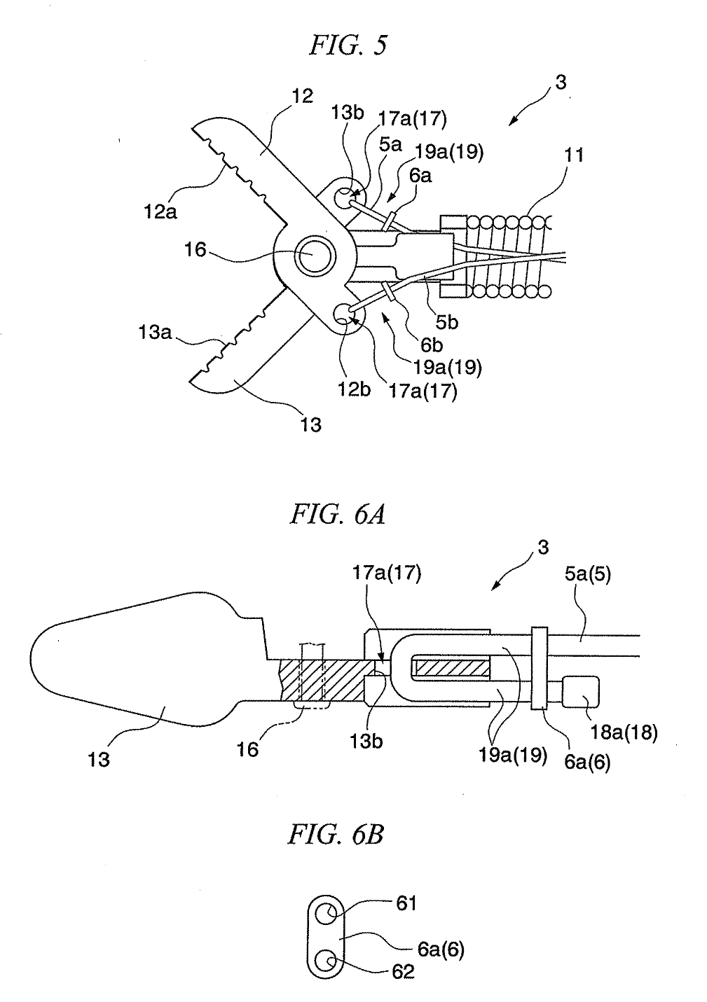

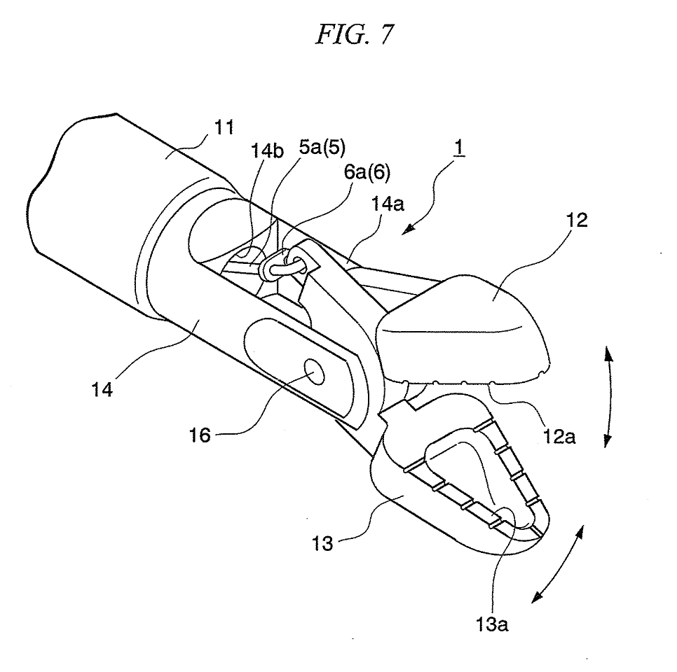

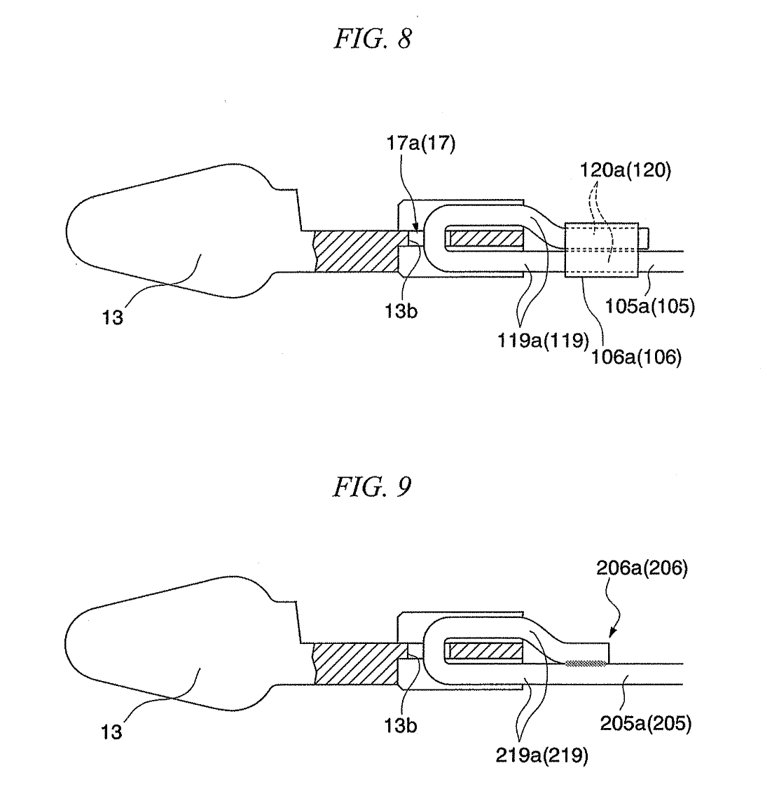

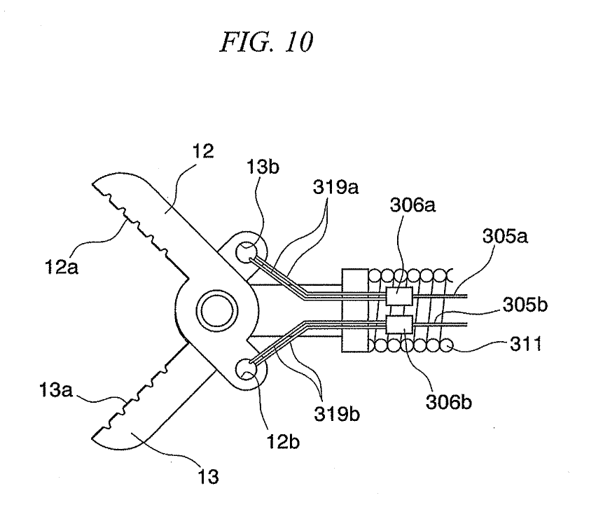

### BRIEF DESCRIPTION OF THE DRAWINGS

[FIG. 1] A configuration diagram of a treatment tool in a first embodiment of the invention, and an endoscope used with the treatment tool.

[FIG. 2] A plan diagram of the same treatment tool.

[FIG. 3] A perspective diagram of the configuration of a treatment part in the same treatment tool.

[FIG. 4] A perspective diagram of the configuration of one part of the same treatment part.

[FIG. 5] A side diagram of a partial cross-section of the configuration of the same treatment part.

[FIG. 6A] A plan cross-sectional diagram of the configuration of one part of the same treatment part.

[FIG. 6B] A plan cross-sectional diagram of the configuration of one part of the same treatment part.

[FIG. 7] A perspective diagram of the same treatment part.

[FIG. 8] A plan cross-sectional diagram of the configuration of one part of a treatment tool in a second embodiment of the invention.

[FIG. 9] A plan cross-sectional diagram of the configuration of one part of a treatment tool in a third embodiment of the invention.

[FIG. 10] A side diagram of a partial cross-section of the configuration of one part of a treatment tool in a fourth embodiment of the invention.

### BEST MODES FOR REALIZING THE INVENTION

### Embodiment 1

A treatment tool according to a first embodiment of the invention will be explained with reference to FIGS. 1 to 7.

FIG. 1 is a configuration diagram of a treatment tool 1 in a first embodiment of the invention, and an endoscope 7 used together with the treatment tool. FIG. 2 is a plan diagram of the treatment tool 1. As shown in FIGS. 1 and 2, the treatment tool 1 includes a long sheath 11 that is inserted into an insertion part 8 of the endoscope 7, and the sheath 11 can be extendably and retractably inserted into a forceps channel 10. A treatment part 3 is provided at the distal end of the sheath 11, and performs treatment to a biological tissue P. The treatment part 3 of this embodiment is a forceps that can open and close.

FIG. 3 is an enlarged perspective diagram of the treatment part 3 in the treatment tool 1. As shown in FIG. 3, the treatment part 3 is provided with a pin 16 that forms a swing shaft, and forceps members 12 and 13 which the pin 16 is inserted into and which can swing about the pin 16. The forceps members 12 and 13 are substantially the same shape and size as each other, and form a pair of mutually opposing gripping parts 12a and 13a that are opened and closed by the swinging of the forceps members 12 and 13.

FIG. 4 is an enlarged perspective diagram of the configuration of one part of the treatment part 3. As shown in FIG. 4, the forceps members 12 and 13 extend from the respective gripping parts 12a and 13a at the distal end to the proximal end, such that they intersect at position of the pin 16. A wire-connecting part is connected to the proximal ends of the forceps members 12 and 13, and includes through holes 12b and 13b which are parallel to the center axis line of the pin 16.

Wires 5 for making the forceps members 12 and 13 swing around the pin 16 are inserted into the through holes 12b and 13b. A wire 5a and a wire 5b connect to the forceps members 12 and 13 respectively.

FIG. 5 is a side diagram of a partial cross-section of the treatment part 3. As shown in FIG. 5, the wires (swing manipulation wires) 5a and 5b are inserted into the through holes 13b and 12b. At both ends of the through holes 13b and 12b, the wires 5a and 5b are bent to the sheath 11 side at right angles and extend parallel to each other, thereby forming parallel parts 19. The parallel parts 19 include parallel parts 19a and 19b for the wires 5a and 5b respectively. The portions of the wires 5a and 5b inserted into the through holes 12b and 13b become rotation shafts 17 (rotation shafts 17a and 17b) that can rotate with respect to the through holes 12b and 13b.

FIG. 6A is a plan cross-sectional diagram of the configuration of one part of the treatment part 3, and shows the wire-connecting part where the wire 5a is connected to the forceps member 13. As shown in FIG. 6A, the portion of the wire 5a inserted into the through hole 13b becomes a rotation shaft 17a that can rotate with respect to the through hole 13b. The center axis line of the rotation shaft 17a and the center axis line of the through hole 13b are formed substantially coaxial and parallel.

As described above, both ends of the rotation shaft 17a are bent to the sheath 11 side at right angles, and extend parallel to each other to become the parallel part 19a. The end of the wire 5a extending to the sheath 11 side is inserted through the sheath 11 and connected to a handle 15 of a manipulating part 2 (see FIG. 1). The handle 15 engages with the manipulating part 2 such that it can move back and forth. A power supply device 4 for supplying high-frequency current to the imaging device 3 via supply electrodes (not shown) electrically connected to the wires 5 can be connected to the manipulating part 2.

Incidentally, the wires 5a and 5b extending into the sheath 11 can be secured collectively inside the sheath 11, or be connected to the handle 15 via a single manipulation wire (not shown).

As shown in FIGS. 5 and 6A, the other ends of the wires 5a and 5b extend between the sheath 11 and the through holes 13b and 12b. Without being inserted into the sheath 11, the other ends of the wires 5a and 5b are supported by insertion into supporting parts (securing members) 6a and 6b. For example, in the forceps member 13, the supporting part 6a supports each wire 5a arranged in parallel at the parallel part 19a.

FIG. 6B is a plan diagram of the configuration of the supporting part 6a. Since the supporting parts 6a and 6b are the same shape and size, only the supporting part 6a will be explained. The supporting part 6a includes two holes (first insertion hole 61 and second insertion hole 62) which the wire 5a can be inserted through. The interval between the first insertion hole 61 and the second insertion hole 62 is equal to the axial lengths of the through holes 12b and 13b of the forceps members 12 and 13.

Preferably, the interval between the first insertion hole 61 and the second insertion hole 62 is slightly longer than the axial lengths of the through holes 12b and 13b. This ensures that the outer peripheral face of the wire 5 is slightly distanced from the proximal-side outer face of the forceps member 13, thereby reducing sliding resistance.

As shown in FIG. 6A, a distal-end bulbous part 18a is provided at an end of the wire 5a arranged between the forceps member 13 and the sheath 11, and has a larger diameter than the outer diameter of the wire 5a. The distal-end bulbous part 18a expands outwardly in the radial direction of the wire 5a to prevent the wire 5a from coming out of the supporting part 6a. In this embodiment, the distal-end bulbous part 18a is formed by fitting and securing a caulking ring. Instead of using caulking, the distal-end bulbous part 18a can be formed by, for example, crushing the end of the wire 5a and expanding it radially outward.

While in this embodiment, the distal-end bulbous part 18a prevents the wire 5a from coming out of the supporting part 6a. Instead of providing the distal-end bulbous part 18, the supporting part 6a can be secured to the outer peripheral face of the wire 5a by caulking, welding, or such like.

In this embodiment, the wire 5 is connected to the forceps member 12 in the same manner as described above. As shown in FIG. 5, the wire 5b is connected by inserting it into the through hole 12b in the same manner as described above, and the configuration includes a rotation shaft 17b, a parallel part 19b, and a supporting part 6b.

FIG. 7 is a perspective diagram of the treatment part 3. As shown in FIG. 7, a distal-end member 14 is disposed between the sheath 11 and the forceps members 12 and 13. One end of the distal-end member 14 is secured by insertion into a distal-end opening in the sheath 11, and another end is fitted on the outer peripheries of both ends of the pin 16. A hole 14b connecting to the opening in the end of the sheath 11 is formed in the distal-end member 14, and enables the wire 5 to be extendably and retractably inserted therein.

The distal-end member 14 supports the pin 16 at two points facing each other with the forceps members 12 and 13 in between, and includes a slit part 14a for ensuring that the forceps members 12 and 13 are not obstructed from swinging about the pin 16.

Functions of the treatment tool 1 of this embodiment with the configuration described above will be explained while referring to FIGS. 1 to 7.

Employing a conventionally known method, the user of the treatment tool 1 inserts the insertion part 8 of the endoscope 7 into an inner cavity or the like of a living biological body and guides it to a treatment target region. As shown in FIG. 1, the user then inserts the treatment tool 1 from the treatment part 3 side into the insertion part 8 of the endoscope 7, and guides it to the biological tissue P that is the treatment target.

The user slides the handle 15 of the manipulating part 2 to the sheath 11 (distal-end) side. Thereby, the wires 5 connected to the optical system 15 are also slide to the distal-end side, and the wires 5a and 5b push against the inner peripheral faces of the through holes 12b and 13b of the respective forceps members 12 and 13. Thus, the through holes 12b and 13b are pushed away from each other, and the forceps members 12 and 13 swing about the pin 16. As a result, the gripping parts 12a and 13a open (see FIG. 7).

As shown in FIG. 1, the user inserts the biological tissue P between the forceps members 12 and 13, and slides the handle 15 toward his hands (toward the proximal-end side). Thereby, the wires 5 connected to the handle 15 also slide toward the proximal-end side. The through holes 12b and 13b are dragged by the wires 5a and 5b, and the forceps members 12 and 13 swing about the pin 16 in the opposite direction to that described above. As a result, the gripping parts 12a and 13a close (see FIG. 7).

When the gripping parts 12a and 13a close, the biological tissue P is gripped between them. The user then performs an appropriate treatment to the biological tissue P, or to another biological tissue near it, or the like. After performing this treatment, the user slides the handle 15 of the manipulating part 2 toward the distal-end side in the same manner as described above, and opens the gripping parts 12a and 13a so as to release the biological tissue P from their grip. After the user removes the sheath 11 and the treatment part 3 from the forceps channel 10 of the endoscope 7, he removes the endoscope 7 from the body cavity, ending this series of manipulations.

As described above, according to the treatment tool 1 of this embodiment, since the dragging force or pushing force acting on the handle 15 is transmitted reliably via the wires 5 to the forceps members 12 and 13 of the treatment part 3, the gripping parts 12a and 13a can be opened and closed.

The supporting parts 6 (supporting parts 6a and 6b) support the parallel parts 19 (parallel parts 19a and 19b) in parallel, and are disposed inside the slit part 14a at a distance from the proximal ends of the forceps members 12 and 13. Therefore, even when the forceps members 12 and 13 swing about the pin 16, the supporting parts 6 do not make contact with the proximal ends of the forceps members 12 and 13. the supporting parts 6 can support the parallel parts 19 of the wires 5a and 5b in parallel while keeping them distance from each other by the length of the through holes 12b and 13b. As a result, the wires 5 are prevented from catching on the forceps members 12 and 13, and sliding resistance is reduced so that the gripping parts 12a and 13a can be opened and closed by optimally sliding the forceps members 12 and 13.

The wires 5a and 5b are folded back at the ends of the through holes 12b and 13b. At the proximal-end sides of the forceps members 12 and 13, the supporting parts 6a and 6b respectively support the wires 5a and 5b so as to prevent them from coming out of the through holes 12b and 13b. Since this configuration is arranged at the proximal-end sides of the forceps members 12 and 13 where there is more space than near the open ends of the through holes 12b and 13b, the workability of assembly can be enhanced.

### Embodiment 2

Subsequently, a treatment tool according to a second embodiment of the invention will be explained with reference to FIG. 8. In each of the embodiments described below, elements of the configuration that are common to the treatment device 1 according to the first embodiment are designated with the same reference numerals and are not repetitiously explained.

FIG. 8 is a plan cross-sectional diagram of the configuration of one part of the treatment part 3 in the treatment tool of this embodiment. As shown in FIG. 8, this embodiment includes wires 105 instead of the wires 5, and includes supporting parts 106 instead of the supporting parts 6. That is, the structure of the connection between the forceps members 12 and 13 and the wires 105 differs from that of the first embodiment.

In this embodiment, as in the first embodiment, the forceps members 12 and 13 are the same shape and size as each other, and the structures of their connections to the wires 105 are the same. Therefore, only the structure of the connection of the wire 105 to the forceps 13 will be explained.

The wire 105 is inserted into the through hole 13b at the proximal-end side of the forceps member 13 as in the first embodiment, and the portion of the wire 105 inserted into the through hole 13b becomes a rotation shaft 17a (17) with respect to the through hole 13b. The wire 105 is bent at a right angle at each end of the opening in the through hole 13b, parallel parts 119 is formed as in the first embodiment.

The parallel parts 119 extend in parallel at a distance from each other corresponding to the length of the through hole 13b, and are mutually adjoined at the proximal-end side of the forceps member 13 to form second parallel parts 120. A tubular supporting part 106 is provided around the outer peripheral faces of the second parallel parts 120.

The supporting part 106 differs from the supporting part 6 of the first embodiment in that the supporting part 106 is formed in a tubular shape that enables both the wires 105 in the second parallel parts 120 to be inserted. The supporting part 106 can be secured by inserting a metal caulk or by inserting a heat shrinkable tube and heat shrinking it, or the supporting part 106 can be secured such that the second parallel parts 120 are pressed into a tube made from elastic material such as rubber or resin so that the supporting part 106 can be supported by the frictional engagement of this tube with the outer peripheral faces of the wires 105. The supporting part 106 is a limiting member for limiting the relative movement of the wires 105 in the second parallel parts 120.

With such a configuration, since the wires 105 adjoining each other in the second parallel parts 120 are secured, the parallel parts 119 are supported in parallel, and the pushing force or dragging force of the wires 105 are optimally transmitted to the forceps member 13. Therefore, as in the first embodiment, the force of the wires acting on the treatment tool can be reliably transmitted.

Also, since the supporting part 106 is a simple tubular member, the cost of manufacturing the treatment tool can be reduced. Moreover, since the wires 105 are adjoined in a bundle in the second parallel parts 120, the second parallel parts 120 can be made narrower than the parallel parts 119.

### Third Embodiment

Subsequently, a treatment tool according to a third embodiment of the invention will be explained with reference to FIG. 9.

This embodiment includes wires 205 instead of the wires 5 and 105, and the structure of the connection of the wires 205 to the forceps members 12 and 13 differs from those in the embodiments described above. The forceps members 12 and 13 are the same shape and size, and the structures of their connections to the wires 105 are also the same.

Only the forceps member 13 will be explained. The wire 205 is inserted into the through hole 13b, and has parallel parts 219 similar to the parallel parts 119 of the second embodiment.

At the proximal-end side of the parallel part 219, one end of the wire 205 extends inside the sheath 11 in the same manner as the wire 5 of the first embodiment. However, the other end of the wire 205 is bent such that at least its tip touches the outer peripheral face of the one end side, and these two portions of the wire 205 are secured by welding them together.

The part where the wires 205 are welded together can be an end face or an end outer circumference at the other end of the wire 205. As described above, since the wires 205 are welded together, the welded part functions as a supporting part 206 for supporting the parallel parts 219 such that they are parallel to each other.

Likewise in this embodiment, since the wires 205 are supported in parallel in the parallel parts 219, as in the embodiments described above, the force of the wires acting on the treatment tool can be reliably transmitted. Furthermore, since the supporting part 206 is made by welding and securing, no members protrude outwardly in the radial direction from the outer peripheral faces of the wires. This enables the supporting part to be made narrower.

### Fourth Embodiment

Subsequently, a treatment tool according to a fourth embodiment of the invention will be explained with reference to FIG. 10.

This embodiment includes wires 305 (wires 305a and 305b) instead of the wires 5, and the structure of the connections between the forceps members 12 and 13 and the wires 305 differs from those of the embodiments described above. Another point of difference from the embodiments described above is that the supporting parts 6 are replaced with supporting parts 306 (supporting parts 306a and 306b).

The forceps members 12 and 13 are the same shape and size, and the structures of their connections to the wires 305 are the same. Therefore, the explanation below describes only the supporting member 306a and the wire 305a connected to the forceps member 13.

The wire 305a is inserted into the through hole 13b of the forceps member 13, and, as in the embodiments described above, a rotation shaft (not shown) and parallel parts 319 are formed. The parallel parts 319 extend to the sheath 11 side, reaching the inside of the sheath 11 via the distal-end member 14.

The ends of the parallel part 319a inside the sheath 11 are inserted into a cylindrical supporting part 306a. The supporting part 306a is fitted to the outer peripheral faces of the wires 305a, and secured at the parallel part 319a such that the wires 305a become parallel. A metal cylinder used in caulking, a heat shrinkable tube, or the like, can be used as the supporting part 306a. The corners of the supporting part 306a can be cut off to ensure that they do not catch on the treatment tool 1 or the distal-end member. At the parallel parts 319b, the supporting part 306b supports the wires 305b in parallel in the same manner as described above.

In this embodiment, as in the embodiments described above, since the parallel parts 319a and 319b are supported in parallel, the force from the wires acting on the treatment tool can be reliably transmitted as in the embodiments described above.

In addition, the supporting parts 306a and 306b are disposed inside the sheath 11 at a distance from the treatment part 3. As a result, the diameter of the section of the treatment part 3 from the forceps members 12 and 13 to the open end of the sheath 11 can be made smaller; or, even if the configuration of the treatment part 3 is made more complex to achieve enhanced functions, it can be prevented that the diameter of the section of the treatment part 3 becomes larger.

Embodiments of the invention have been described in detail with reference to the drawings. However, the specific configuration is not limited to these embodiments, and includes design modifications and the like that do not depart from the scope of the main points of the invention.

### INDUSTRIAL APPLICABILITY

According to the treatment tool of the invention, by making a treatment part swing by dragging/pushing parallel parts that extend from both ends of a rotation shaft, the force applied from the wires to the treatment tool can be reliably transmitted.

### REFERENCE NUMERALS

1 TREATMENT TOOL
2 MANIPULATING PART
3 TREATMENT PART
5, 5A, 5B, 205, 305, 305a, 305b WIRES (SWING MANIPULATION WIRES)
6, 6a, 6b, 106, 206, 206a, 206b, 306, 306a, 306b SUPPORTING PARTS (SECURING MEMBERS, LIMITING MEMBERS)
P BIOLOGICAL TISSUE
11 SHEATH
12, 13 FORCEPS MEMBERS
12a, 12b GRIPPING PARTS
14 DISTAL-END MEMBER
14a SLIT PART
17a, 17b ROTATION SHAFT
19, 19a, 19b, 119, 219, 319, 319a, 319b PARALLEL PARTS
61 FIRST INSERTION HOLE
62 SECOND INSERTION HOLE
105 WIRE
120 SECOND PARALLEL PART

1. A treatment tool comprising:
a treatment part that can swing about a predetermined swing axis in order to perform treatment to a biological tissue;
a plate-like wire-connecting part provided at said treatment part, the wire-connecting part having a predetermined thickness and a through hole formed therein in its thick direction;
a linear swing manipulation wire, on one end side thereof are formed a first contacting part that is inserted into the through hole and contacts one face of the wire-connecting part in the thickness direction thereof, and a second contacting part that contacts another face of the wire-connecting part in the thickness direction thereof;
a securing member, secured on the one end side of the swing manipulation wire, that holds the first contacting part and the second contacting part such that the first contacting part slides along the face, and said second contacting member slides along the other face; and
a manipulating part, provided at another end of the swing manipulation wire, that extends and retracts the swing manipulation wire to make said treatment part swing.

2. The treatment tool according to claim 1, wherein
the swing manipulation wire is folded back by bending it at both ends of the through hole; and
a first insertion hole which either one of the folded-back sections of the swing manipulation wire is inserted into, and a second insertion hole which another of the folded-back sections of the swing manipulation wire is inserted into, are formed in the securing member.

3. The treatment tool according to claim 1, wherein said securing member is fitted over said swing manipulation wire, and supports said swing manipulation wire.

4. The treatment tool according to claim 1, wherein the treatment part includes a pair of gripping parts that open and close by swinging about the swing shaft.

5. The treatment tool according to claim 1, further comprising:
a sheath which the swing manipulation wire is extendably and retractably inserted into; and
a distal-end member that is provided between the sheath and the treatment part and connects a distal end of the sheath and the treatment part;
and wherein
the distal-end member includes a slit part on at least the distal-end side; and
the securing member supports the swing manipulation wire on an inner side of said slit part.

6. The treatment tool according to claim 1, further comprising:
a sheath which the swing manipulation wire is extendably and retractably inserted into; and
a distal-end member that is provided between the sheath and the treatment part and connects a distal end of the sheath and the treatment part;
and wherein
the distal-end member includes a slit part on at least the distal-end side; and
the supporting part supports said wire on an inner side of said slit part.
